(19) United States
(12) Patent Application Publication
(10) Pub. No.: US 2003/0055315 A1
(43) Pub. Date: Mar. 20, 2003

(54) Method and apparatus for endoscope system

(76) Inventors: Dominick L. Gatto, Branford, CT (US); James Bonneville, Worcester, CT (US); Michael F. Jacene, Southbridge, MA (US)

Correspondence Address: Ki O, Joseph A. Micallef, ARNOLD & PORTER, 555 12th Street, N.W., Washington, DC, 20004-1206, US

(21) Appl. No.: 09/951,545

(22) Filed: Sep. 14, 2001

(51) Int. Cl. A61B 1/00

(52) U.S. Cl. 600/114; 600/104

(57) ABSTRACT

A three part microendoscope system including a cannula, a dilator, and endoscopic insert for detection of breast cancer are disclosed. To facilitate surgical entry of an endoscope system through the sphincter of a breast, a special dilator is used with an endoscope so that the endoscope can more readily pass through tortuous passageways. In accordance with the present invention, a semi-rigid endoscope can be advanced through the sphincter in the nipple using a nipple dilator. The microendoscope uses a special lumen tube that is usable with various insertable tools that allows for minimal invasive damage to surrounding cells. Insertable tools to irrigate, lavage, or perform biopsies on targeted areas of the breast are disclosed. A novel video coupler having a predetermined focus and orientation to be used with the present system is also disclosed. A method of using the three part microendoscope system and its various insertable tools are disclosed. A method of manufacturing the video coupler is disclosed.

The present invention incorporates by reference in its entirety U.S. patent application Ser. No. 08/155,748, filed Nov. 23, 1993 by Tony Petitto and Stanislaw Loth, for “Technique for Depth of Field Viewing of Images With Improved Clarity and Contrast,” now U.S. Pat. No. 5,400,177. It also incorporates by reference Ser. No. 08/115,748, filed Nov. 23, 1993, which is now U.S. Pat. No. 5,400,177, and Ser. No. 09/115,809, which is now abandoned, and Ser. No. 09/501,313, which is a continuation of Ser. No. 09/006,894, filed Jan. 14, 1998, and Ser. No. 09/524,746, filed Mar. 14, 2000.

### BACKGROUND OF THE INVENTION

(1) Field of the Invention

This invention relates generally to a medical apparatus and its medical use for diagnosing, exploring, and treating abnormalities in internal body tissue. In particular, the invention is directed to a three part endoscope system having a cannula, semi-rigid microendoscope, and a dilator. The present invention allows the use of a specifically designed microendoscope to view body tissues without causing severe damage to surrounding tissue. The microendoscope system allows for easy entry into small body orifices, and yet it produces high resolution images. In one embodiment, the invention allows a physician to view targeted cells in the breast of a patient by introducing the microendoscopic system through the sphincter of the breast nipple. The physician can perform diagnostic tests by using a nipple dilator that facilitates the guidance of microendoscope to its desired location. The microendoscope allows for imaging of the desired location with minimal invasive damage to the surrounding tissues. The invention can also be used to lavage internal tissues, explore certain targeted areas of tissue, perform biopsies, deliver medication or other types of materials to a targeted area while guiding the physician with microendoscopic images. The invention is manufactured using a novel predetermined orientation design that offers unique advantages for the physicians during the surgical procedure.

(2) Discussion of the Prior Art

Many forms of endoscopes are known in the art. They generally comprise an elongated multi-lumen tube having a proximal end and a distal end. One lumen usually contains an optical fiber or bundle of such fibers for transmitting light from a light source. The other lumen typically contains further optical fibers for transmitting the illuminated image from the distal end of the endoscope to the proximal end of the endoscope. This image is usually transmitted to an eyepiece or a viewing device. An endoscope will typically contain a lumen comprising at least one working channel by which flushing liquid or any other liquid, medication, or substance can be delivered to the distal end of the endoscope so that it can reach a targeted location in the body tissue.

Various techniques can be used to place the endoscope at a desired location in the patient. These techniques, however, have been problematic in the past. For example, a physician can use a guidewire to assist the cannula or catheter in order to find the desired location in the body tissue. Once the catheter is positioned properly, the guidewire may be withdrawn from the catheter and thereafter an endoscope inserted through a lumen of the catheter. This technique can be disadvantageous, because the guidewire guidance into body tissue requires arbitrary trial and error guidance and the system does not allow a doctor to navigate the guidewire to targeted cells by using images from the endoscope. Another technique uses a monorail system in which the endoscope runs along the same axis as the guidewire, which then allows the operator to track and place the catheter at the desired location. Both of these systems lack the capability to guide a physician to targeted areas without having the physician at least make some educated guesses as to where that targeted area may be. Our current invention removes some of the guess work by allowing physicians to make initial entries into the human body using a cannula, dilator, and an insertable endoscope so that physicians may guide the device to targeted areas using images collected from the endoscope.

### OBJECTS

It is accordingly a principal object of the present invention to provide a combination of instruments including a cannula, endoscope, and a dilator, which may be utilized in such procedures to achieve the respective benefits of both rigid and semi-rigid endoscopes.

Another object of the invention is to provide the combination of a dilator and a endoscope where the dilator has a size permitting the endoscope to be passed through narrow, small body orifices, such as the nipple sphincter, with minimal invasive damage to surrounding cells.

Still another object of the invention is to provide an endoscope system that is sized to enter the sphincter of the nipple and capable of guiding a physician to locate diseased cells in the breast.

Still another object of the invention is to provide a minimally invasive endoscope system that produces a high quality image of the desired locations on a viewing device or eyepiece using a novel design that allows for a reduced number of illumination fibers.

Still another object of the invention is to provide a method of performing an endoscopic procedure in a new, minimally invasive procedure allowing the physician to locate diseased cells efficiently.

Still another object of the invention is to provide an endoscope lumen cannula that is sized to fit various insertable tools, including biopsy tools, lavaging tools, or other surgical tools.

Still another object of the invention is to provide a new technique of performing surgical diagnostic tests on body tissue using a three system microendoscopic system that causes minimal damage to surrounding tissue.

### SUMMARY OF THE INVENTION

In accordance with the present invention, there is disclosed a three part microendoscope system comprising a cannula having a tubular member with a predetermined length, outside diameter and inside diameter, said cannula having a proximal end, a distal end, and a lumen extending therebetween, said cannula having a cross section area of the lumen, a dilator having a wire frame with a predetermined length greater than said length of said cannula, an outer diameter that has a cross-sectional area that is smaller than said cross section area of the lumen such that said dilator can slide longitudinally and coaxially through said cannula, a scope assembly comprising a tubular member with a certain length, outside diameter and inside diameter, said tubular member having a proximal end, a distal end, and a lumen therebetween, said scope assembly having a scope operatively connected to said proximal end of said tubular member, and optical fibers extending from said scope to said distal end of said tubular member, said lumen containing optical fibers for transmitting light from said proximal end of said cannula to said distal end of said cannula, and a hub attached to said cannula having a bore that is in operatively connected to said lumen of said cannula.

There is also disclosed an endoscope system comprising a cannula with a cross-sectional design that allows for empty areas for the transportation of irrigation solutions or materials used for the purposes of performing lavages, biopsies, or diagnostic testing.

A novel coupler device is disclosed for coupling an endoscope to a camera or to a display means comprising a housing having a light source and a light path that is transferable to the distal end of the endoscope, said housing having a connection means, a housing with connection means that fit said connection means of said housing so that there is only one orientation by which they can connect and produce a fixed focus, lens means mounted within the housing for magnifying an image from the endoscope in order to provide a magnified image, and means for aligning the housing and the hub in a predetermined orientation so that it produces an fixed focus image.

There is also disclosed a method of manufacturing an endoscopic system comprising the steps of: making a coupler to connect the endoscope to the video viewing device wherein the coupler is polished to produce flat surfaces such that the image guide of the housing is flush and flat against the front planar surface of the housing so that focal point is fixed between the transfer of image from the hub to the housing.

There is also disclosed a method of performing an endoscopic procedure that uses a removable dilator with a cannula lumen tube and a removable endoscope insert so that the physician can manipulate the passageway of the device with accuracy causing minimal invasive damage to surrounding cells.

There is also disclosed various types of insertable tools for the cannula including a twist window system comprising a first twist window tubular member with a certain length, outside diameter and inside diameter, said first twist window tubular member having a proximal end, a distal end, and a uniform lumen therebetween, said twist window tubular member being sized to fit within said cannula coaxially, a second twist window tubular member having a certain length, outside diameter and inside diameter, said second twist window tubular member being sized to fit within and insertable within said first twist window tubular member, said first twist window tubular member having a first cutting surface created by an aperture or window, said second twist window tubular member having a second cutting surface created by an aperture or window.

There is also disclosed a scissors-like insert having a first scissors tubular member with a certain length, outside diameter and inside diameter, said first scissors tubular member having a proximal end, a distal end, and a uniform lumen therebetween, said scissors tubular member being sized to fit within said cannula coaxially, a second scissors tubular member having a certain length, outside diameter and inside diameter, said second scissors tubular member being sized to fit within and insertable within said first scissors tubular member, said first scissors tubular member having a cutting surface created by an edge at said distal end of said first tubular member, said second tubular member having a second cutting surface created by an aperture or window, said first and second cutting surfaces having a predetermined locations such that when second scissors tubular member is inserted within said first tubular member, said first and second cutting surfaces are aligned,

There is also disclosed a tissue abrader insert comprising a tissue abrader first tubular member with a certain length, outside surface, outside diameter and inside diameter, said tissue abrader first tubular member having a proximal end, a distal end, and a uniform lumen therebetween, said tissue abrader tubular member being sized to fit within said cannula coaxially, said tissue abrader tubular member having burrs created by small protrusions on said surface of said tissue abrader first tubular member, said protrusions having small openings and cutting edges sized to cut and fit small tissue cells,

There is also disclosed a spring grasp insert comprising a spring grasp first tubular member with a certain length, outside surface, outside diameter and inside diameter, said spring grasp first tubular member having a proximal end, a distal end, and a uniform lumen therebetween, said spring grasp tubular member being sized to fit within said cannula coaxially, a grasp tool comprising an elongated member with a proximal end and a distal end, said grasp tool having at least two gripping jaws capable of closing and opening and collecting targeted tissue cells, said gripping jaws being located at the distal end of said grasp tool, and said gripping jaws having a predetermined shape in an open position.

And there is disclosed a laser insert comprising a laser tubular member with a certain length, outside surface, outside diameter and inside diameter, said laser tubular member having a proximal end, a distal end, and a uniform lumen therebetween, and said tubular member being sized to fit within said cannula coaxially, a laser transmitting fiber extending from said proximal end to said distal end capable of transmitting various types of laser light to the distal end of said laser insert system.

### DESCRIPTION OF THE DRAWINGS

Further details, objects and advantages of the invention will become apparent to those skilled in the art from the following detailed description of a embodiment in which like numerals in the several views refer to corresponding parts.

FIG. 1 is an illustration of the endoscope system including the endoscope insert, video coupler, cannula and the dilator.

FIG. 2 is an illustration of the cannula and the dilator.

FIG. 3 is an illustration of the endoscope insert and the video coupler.

FIG. 4A is an illustration of the cross section of a prior art working channel.

FIG. 4B is an illustration of the cross section of a working channel embodied in the current invention.

FIG. 5A is an illustration of the dilator body and dilator distal end.

FIG. 5B is an illustration of a magnified version of the dilator distal end.

FIG. 6 is an illustration of the coupler showing the front housing and the rear housing.

FIG. 7 is an inner exposed illustration of the coupler showing the back face of the housing and the front face of the housing.

FIG. 8 is a spread-out illustration of the coupler components.

FIG. 9 is an illustration of a twist window tool used for biopsies and insertable into the cannula.

FIG. 10 is an illustration of a scissors-like tool used for biopsies and insertable into the cannula.

FIG. 11 is an illustration of a tissue abrader tool used for biopsies and insertable into the cannula.

FIG. 12 is an illustration of a spring grasp tool used for biopsies and insertable into the cannula.

FIG. 13 is an illustration of a alternative spring grasp tool with bendable elbows used for biopsies and insertable into the cannula.

FIG. 14 is an illustration of an excimer laser tool used for biopsies and insertable into the cannula.

### DESCRIPTION OF THE EMBODIMENT

Although the present invention is described below in connection with specific embodiments, it will be appreciated that the invention is not limited to the described embodiments.

In endoscopic procedures, a video image is transmitted directly from the inside of the patient's body to a viewing device, such as a video monitor. Of particular importance is microendoscopic procedures that are viewed through micro-optic fiber image conduits. In breast exploration, microendoscopic procedures utilize scopes sized to be minimally invasive to breast tissues. These procedures utilize fiber optic bundles that range in diameter from 0.2 mm to 3.0 mm. Such procedures eliminate the need to open large operation areas and allow one to reach into and see inside very small and narrow body ducts. It reduces the patient's trauma, stress, danger of infection, allowing the patient in most cases to recover quickly.

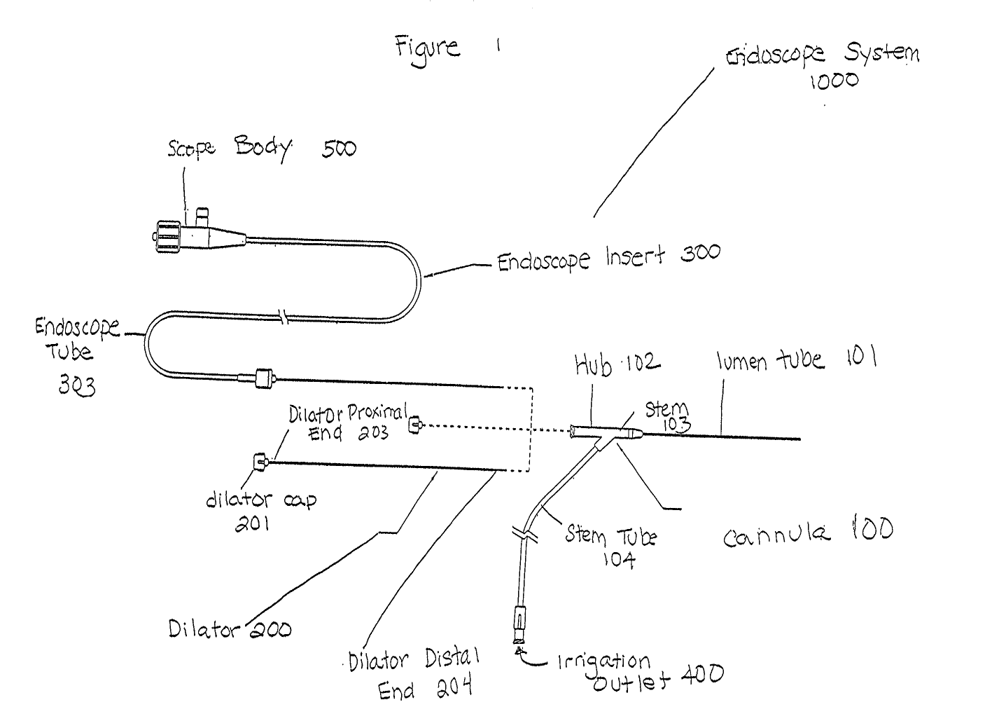

Referring now to FIG. 1, the current endoscope system 1000 is comprised of three main components: the cannula 100, the dilator 200, and the endoscope insert 300. Various other useful tools are insertable into the cannula 100 instead of the endoscope insert 300. The cannula 100 has a hollow, elongated lumen tube 101 through which the dilator 200 or endoscope insert 300 can be inserted. The elongated lumen tube 101 is operatively connected to hub 102, which is operatively connected to stem line 103. Stem line 103 is fluidly connected to an irrigation outlet 400 to deliver various irrigation solutions during a surgical procedure. The dilator 200 is capable of sliding coaxially into cannula 100. The endoscope insert 300 and the various other inserts are also capable of sliding coaxially into cannula 100. The endoscope insert 300 may be connected to a viewing device such as an eyepiece or a monitor. In accordance with the current invention, the endoscope insert 300 has a scope body 500 to relay images to a video monitor (not shown).

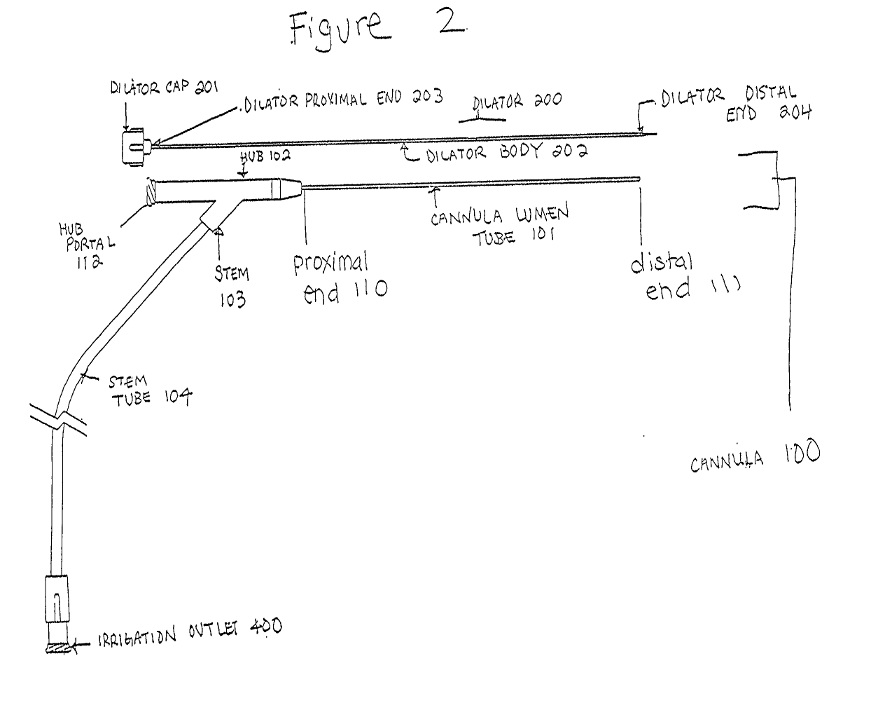

Referring next to FIG. 2, there is illustrated a side view of the cannula 100 and dilator 200. The dilator 200 is comprised of a dilator cap 201 and the dilator body 202. The dilator 200 is capable of fitting into the hollow lumen tube 101 coaxially. The lumen tube 101 contains a proximal end 110 and a distal end 111. The lumen tube 101 can be of various length depending on the need for a particular surgical procedure. For breast exploration, the lumen tube 101 can be about 10 cm long with an outer diameter of approximately 0.9 mm and an inside diameter of about 0.8 mm (0.032 inches). For some procedures, it is more appropriate to have a longer rigid guide, such as one approximately 30 cm long. Lumen tube 101 alternatively can be a semi-rigid tube made of semi-rigid plastic or some other suitable material having a length between 3 cm and 30 cm.

Various components including the endoscope insert 300, the nipple dilator 200, the twist window tool 1100, the scissors tool 1200, the tissue abrader tool 1300, the spring grasp tool 1400, and other tools are interchangeably insertable and attachable to the lumen tube 101. In one embodiment, the physician can first introduce the device into the patient using the cannula 100 and the dilator 200. Each of the insertable tools may have a cap or a connecting device at its respective proximal end. In other words, when the interchangeable component is inserted into the lumen tube 101, the component may have a connection means at its proximal end. The cannula 100 may have a countering locking component on its hub portal 112. As shown in FIG. 1, the dilator 200 has a dilator cap 201 attached to the dilator proximal end 203. This dilator cap 201 is connectable to hub portal 112 by conventional connector means. For example, the connecting components can lock using a luer connector or luer slip although there are many other screw-type connectors or snap-like connectors that would be adequate.

The connection for the components to the cannula serves a few different purposes. First, the interchangeable components must be securely fastened to the lumen tube 101 so that they do not come loose during a surgical procedure. Second, when the locking component is locked or secured to the lumen tube 101, it assures that the component remains in the center of the lumen tube 101 to create an irrigation channel around the perimeter of the insertable component. Third, it assures that the distal end of the endoscope is aligned properly, such as the need for the tapered distal end of dilator 200 to exceed the length of the lumen tube 101 so that it can facilitate the system through the a natural or manmade orifice in the human body, such as the sphincter of the nipple.

The lumen tube 101 can be made from various different materials, such as stainless steel or a suitable plastic such as polycarbonate, polyamide, or a polyester that may or may not be reinforced. Stainless steel has certain advantages because it provides rigidity in an easily manufactured narrow tube. The rigidity is necessary to maneuver the device through various tissues while conducting the surgical procedure.

The nipple dilator 200 contains a long needle-like body that has a length slightly longer than lumen tube 101. For example, when used in breast exploration, if the lumen tube is 10 cm long, the dilator body 202 would be slightly longer than 10 cm. When the dilator body 202 is placed inside the lumen tube 101 coaxially such that the lumen cap is flush with the hub portal 112, the dilator distal end 204 extends past the distal end 111 of the cannula by a predetermined length exposing the dilator tapered end. The dilator body 202 can be made from various metals or plastic materials such as polycarbonate, polyamides, polyester, or similar materials. As contemplated in this invention, the dilator body 202 is made from stainless steal because of its rigidity and its ease of manufacture and use.

The dilator distal end 204 may have various shapes depending on the intended use of the invention. In one embodiment, the distal end 111 of the dilator is tapered to a narrow point such that it can pass through small body orifices, such as the nipple sphincter. Design, shape, and pattern for the distal end section may be specific for particular surgical use. For example, the tapered distal end section may be 2 cm long or only a couple of mm long depending on whether a long or short tapered end is needed.

The hollowed section of the lumen tube 101 is longitudinally uniform. The outside diameter of the lumen tube 101 may vary depending upon the intended use of the system. The outside diameter of the tube 101 is preferably no larger than 1.2 mm if the device is to be used in breast exploration. The outside diameter will vary in a range from 0.5 mm to 1 cm depending on the surgical need, preferably 0.5 mm to 1 mm for breast exploration. There is a need to make the outside diameter larger for some surgical procedures while a small diameter would be beneficial for other procedures, such as breast exploration. The internal diameter of lumen tube 101 may vary depending on the thickness of the walls of the lumen tube, but the diameter of the internal diameter is larger than the outside diameter of the various inserts. The outside diameter of the dilator body 202 must be smaller than the internal diameter of the lumen tube such that the various inserts can slide into lumen tube 101 coaxially.

The hub 102 has a hollow internal section that is operatively connected to lumen tube 101 and hub portal 112. The hub 102 may be made from various materials, but generally it is made from molded plastic. The hub 102 also contains an outlet to an irrigation outlet 400 through a y-connector attachment in stem 103. Stem 103 is in fluid communication with stem tube 104. When irrigation is necessary at the end of the distal end 111, a physician may introduce various irrigation solutions and medication through the stem 103. For example, a physician often uses saline to irrigate targeted cell areas. Through the irrigation channel, liquids, medication, or gaseous substances may be injected into the irrigation channel for delivery to the targeted cell area. Such liquids can include water, saline, anesthetics, or antiseptics. The injection can occur through the operation of a syringe or other similar instrument coupled to an irrigation port in the irrigation outlet 400. Moreover, the irrigation channel can also be used as a port through which a laser, such as an excimer laser, could be utilized. In that situation, the laser is inserted through the irrigation channel or the working channel when components are removed from the lumen tube 101. The laser can be in the form of an insertable tool that is lockable onto the lumen tube 101. Excimer laser inserts may be particularly useful, because laser light has the capability to clear blocked passageways.

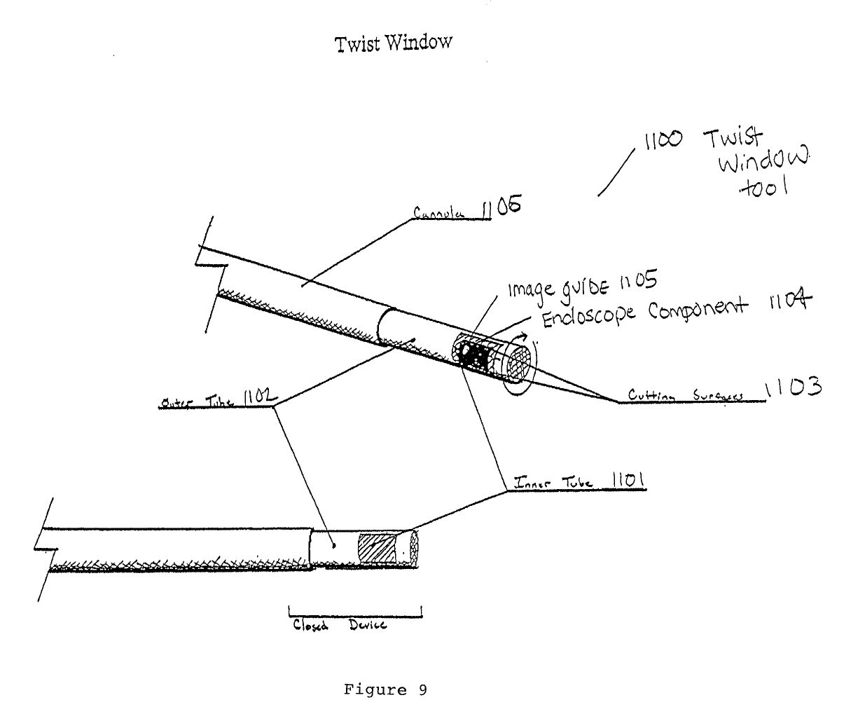

Additional devices can be inserted into the cannula 100. For example, physicians often need DNA samples or cell samples from the infected targeted tissue area. Many interchangeable components can be inserted into the lumen tube 101 for various other purposes. As illustrated in FIG. 9, a twist window tool 1100 is disclosed both in the open and closed positions for purposes of performing a biopsy. The twist window tool 1100 includes a cannula 1106 that utilizes two cutting surfaces between the inner tube 1101 and outer tube 1102. The cutting surfaces are windows or apertures on the outer and inner tubes. The windows may have various sizes and shapes depending on the need for the targeted cells. The windows may have a small dimension to only fit a small number of targeted cells or large enough to collect a large number of cells. The twist window tool 1100 has a window of various shape, design and size on each of its outer tube 1102 and inner tube 1101. In use, each of the windows 1103 line up next to each other in a fashion that allows for the twisting of one tube with respect to the other such that pinching of the tissue cells results. The tissue cells are loosened and trapped inside the tube, which is removed by simply removing the insert tool and extracting the tissue from it.

The twist window 1100 includes an endoscope component 1104 extending coaxially down the center of the cannula 1106. In one embodiment, the functioning endoscope component 1104 aids the physician in collecting targeted cells by sending images from the distal end of the twist window back to the physician. The outer tube 1102 is sized to fit various types of fibers coaxially in cannula 1106, which allows for various sized endoscopes to extend coaxially in cannula 1106.

In practice, the twist window 1100 is an insert that is insertable into cannula 1106. In the three part endoscopic system, cannula 1106 is lumen tube 101. The current system allows a physician to collect targeted cells without having to remove lumen tube 101 from the patient. The use of lumen tube 101 without having to pull it out and reenter with another biopsy tool allows for minimal invasiveness to surrounding tissue. Once the twist window 1100 is in a desired location, an image guide 1105 is transferred from the distal end of the twist window 1100 to a viewing device controlled by a physician. The physician would be capable of viewing the different types of cells at the distal end of the twist window 1100 before using the twist window 1100 to collect the targeted cells.

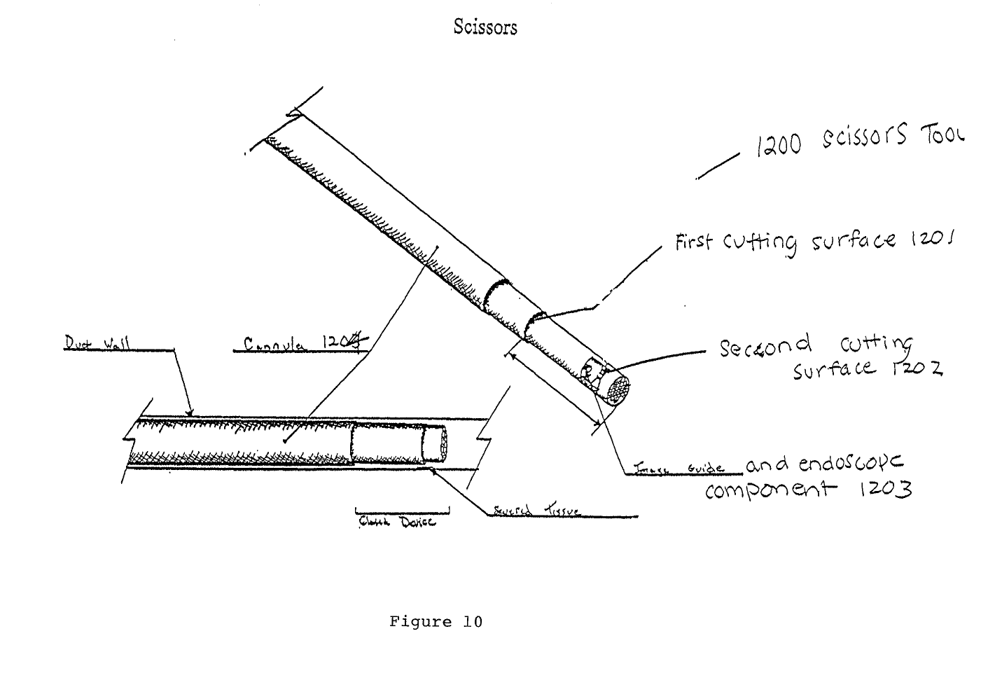

Now referencing FIG. 10, there is illustrated a scissors-like tool 1200 in both its open and closed positions for purposes of performing a biopsy. The scissors tool utilizes a cannula 1204 and two cutting surfaces, the first cutting surface 1201 and the second cutting surface 1202. The thickness of the tubing can pinch the tissue cells between its two edges to trap the cells. In use, the physician will pull the inner tube out towards the outer one cutting and collecting tissue. The scissors-like tool 1200 is then removed and the cells are extracted.

The scissors tool 1200 includes an endoscope component 1203 extending coaxially down the center of the cannula 1204. In one embodiment, the functioning endoscope component 1203 aids the physician in collecting targeted cells by sending images of the distal end of the window created by the second cutting surface 1202 back to the physician. The cannula 1204 is sized to fit various types of fibers coaxially in cannula 1204, which allows for various sized endoscopes to extend coaxially in cannula 1204.

In practice, the twist window 1200 is an insert that is insertable into cannula 1204. In the three part endoscopic system, cannula 1204 is lumen tube 101. The current system allows a physician to collect targeted cells without having to remove lumen tube 101 from the patient. The use of lumen tube 101 without having to pull it out and reenter with another biopsy tool allows for minimal invasiveness to surrounding tissue. Once the twist window 1200 is in a desired location, an image guide 1105 is transferred from the distal end of the twist window 1100 to a viewing device controlled by a physician. The physician would be capable of viewing the different types of cells at the distal end of the twist window 1200 before using the twist window 1100 to collect the targeted cells.

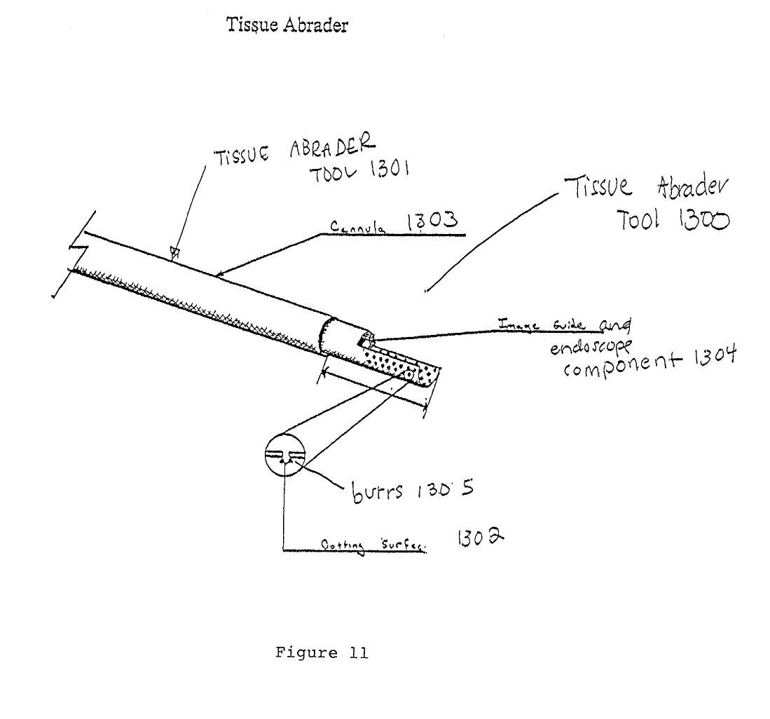

Referring next to FIG. 11, there is illustrated a tissue abrader tool 1300. It utilizes tube 1301 with the end cut away in order to allow for cutting holes to be punched through one wall of the tube. The cutting holes create a cutting surface 1302, which scrapes cells off of tissue walls allowing those cells to be retrieved out of the ducts and analyzed. The cutting holes may be punched through one wall of the tube creating burrs 1305 on the outer surface of the tube wall. In one embodiment, the cutting holes are protrusions on the outer surface wall having small openings and cutting edges sized to cut and fit small tissue cells.

In use, the tissue abrader tool 1300 can be inserted into the cannula 1204 allowing the cutting surface to be exposed at the distal end 111. Once the targeted area is identified, the tissue abrader tool 1300 is rubbed against the body area loosening the cells and tissue using a back and forth motion, as well as a twisting motion.

The tissue abrader 1300 includes an endoscope component 1304 extending coaxially down the center of the cannula 1303. In one embodiment, the functioning endoscope component 1304 aids the physician in collecting targeted cells by sending images of the distal end of the tissue abrader back to the physician. The cannula 1303 is sized to fit various types of fibers coaxially in cannula 1303, which allows for various sized endoscopes to extend coaxially in cannula 1303.

In practice, the tissue abrader 1300 is an insert that is insertable into lumen tube 101, which allows a physician to collect targeted cells without having to remove lumen tube 101 from the patient. The use of lumen tube 101 without having to pull it out and reenter with another biopsy tool allows for minimal invasiveness to surrounding tissue. Once the tissue abrader 1300 is in a desired location, an image guide 1304 is transferred from the distal end of the tissue abrader 1300 to a viewing device controlled by a physician. The physician would be capable of viewing the different types of cells at the distal end of the tissue abrader 1300 before using the tissue abrader 1300 to collect the targeted cells.

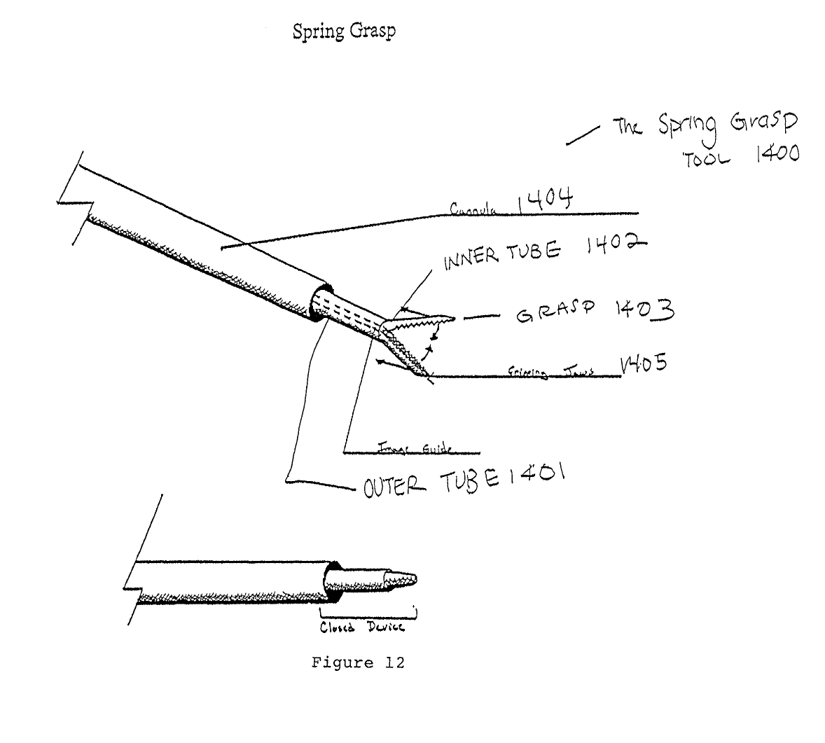

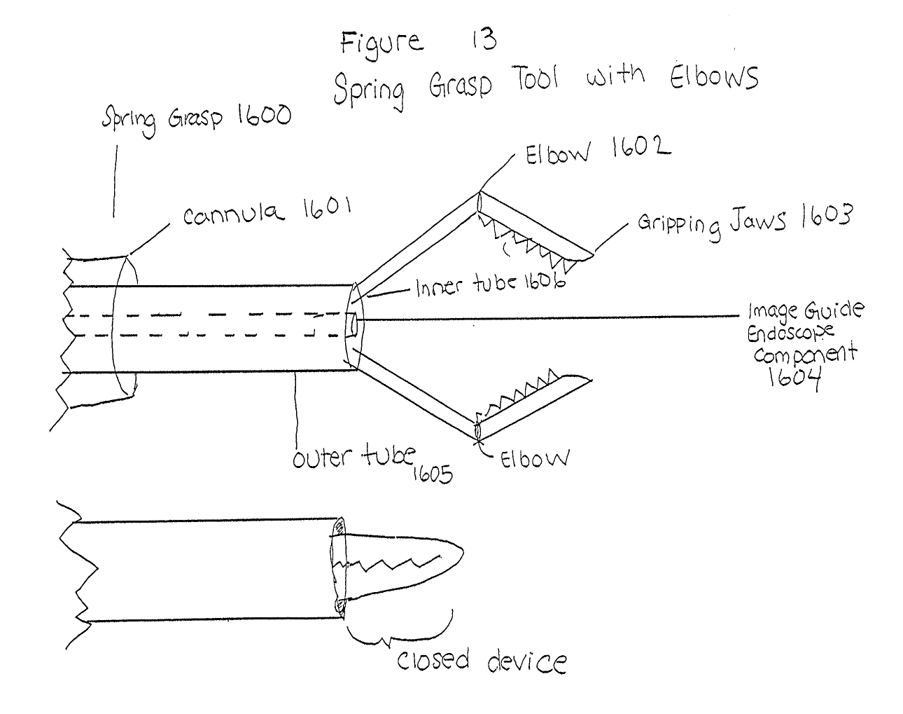

Referring next to FIG. 12, there is illustrated a spring grasp tool 1400. The spring grasp tool 1400 can be made from various materials, but in one embodiment, it is made from a memory metal, such as Nitinol, magnesium alloy, or some other memory metal. Shape Memory Alloys (SMA's) are novel materials that have the ability to return to a predetermined shape when heated. When an SMA is cold, or below its transformation temperature, it has a very low yield strength and can be deformed quite easily into any new shape—which it will retain. When the material is heated above its transformation temperature, however, it undergoes a change in crystal structure that causes it to return to its original shape. If the SMA encounters any resistance during this transformation, it can generate extremely large forces. The most common shape memory material is an alloy of nickel and titanium called Nitinol (50% Ni, 50% Ti). This particular alloy has good electrical and mechanical properties, long fatigue life, and high corrosion resistance. The memory metal allows for a spring force to hold the grasp open under its own forces.

There is disclosed an outer tube 1401 and an inner tube 1402. The outer tube 1401 is used to close the grasps when the inner tube is pulled back into it. The grasp 1403 is inserted into the outer tube 1401. The outer tube is inserted into cannula 1404. In the three part endoscopic system, the cannula 1404 is the lumen tube 101. By pulling the device back into its outer tube 1401, the device closes, which locks down on the tissue and effectively rips the tissue out of the tissue lining. The spring grasp tool 1400 is then removed from the lumen tube 101 and the tissue cell samples can easily be collected.

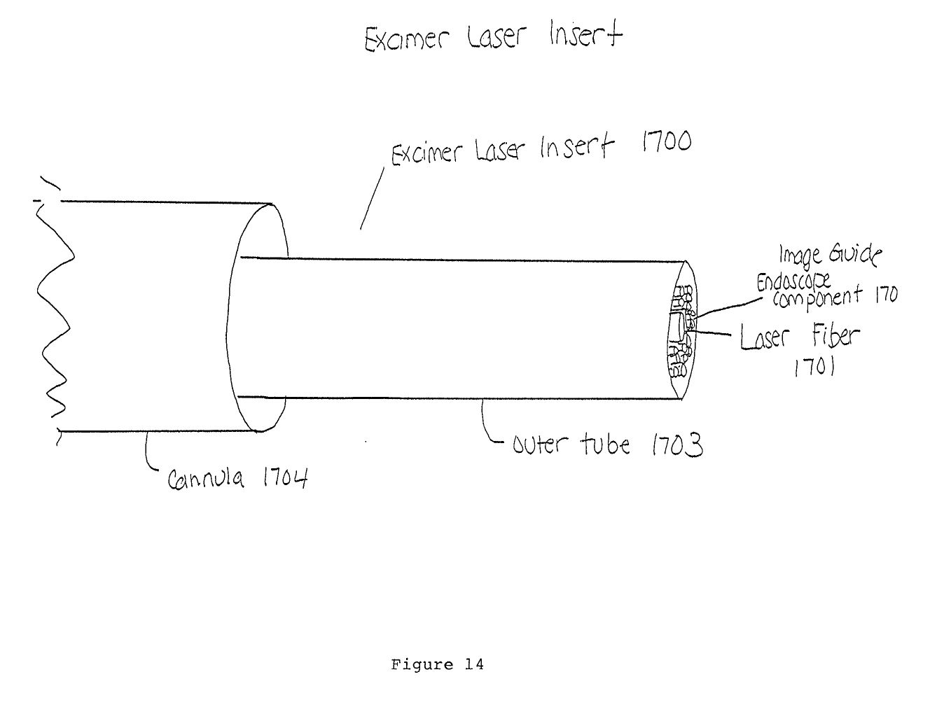

In an alternative embodiment, as shown in FIG. 13, the spring grasp tool has similar features to the embodiment of FIG. 12 except that the gripping jaws 1603 have a bendable elbows 1602. The bendable elbows 1602 provide a better angle to grasp cell tissue by grasping the cells from opposing sides. They also provide proper leveraging for a stronger grip of the tissue and for facilitated removable. When the inner tube 1606 is withdrawn from outer tube 1605, the bendable elbows 1602 bend at its respectable elbows when pressed against the outer tube 1605. The bendable elbows straighten to conformably fit into outer tube 1605 where the whole system can be removed from the patient with the collected cells. Now referencing FIG. 14, there is illustrated a excimer laser insert 1700. The laser tool extends coaxially at the center of outer tube 1703. The excimer laser initiates at the proximal end of the laser insert where there is some source of laser light. Conventional excimer lasers or other type of lasers may be used. The laser light is transferred onto an optical fiber that is capable of carrying laser light. Special optical fibers are available that are capable of transferring the laser light to the distal end of excimer laser tool 1700. The laser is useful for any type of conventional laser uses in surgical fields, including removing unwanted cells, opening blocked passageways, and cauterizing or manipulating various tissue cells of choice.

The laser insert 1700 includes endoscope component 1702 extending coaxially down the center of the outer tube 1703. In one embodiment, the functioning endoscope component 1702 aids the physician in collecting targeted cells by sending images of the distal end of the laser insert 1700 back to the physician. The outer tube 1703 is sized to fit various types of fibers coaxially, which allows for various sized endoscopes and optionally additional illumination fibers 1705 to extend coaxially in cannula 1704.

In practice, the laser tool 1700 is an insert that is insertable into cannula 1704. In the three part endoscopic system, cannula 1704 is lumen tube 101. The current system allows a physician to manipulate targeted cells without having to remove lumen tube 101 from the patient. The use of lumen tube 101 without having to pull it out and reenter with another tool allows for minimal invasiveness to surrounding tissue. Once the laser tool 1700 is in a desired location, an image guide is transferred from the distal end of the laser tool 1700 to a viewing device controlled by a physician. The physician would be capable of viewing the different types of cells at the distal end of the laser tool 1700 before using the laser tool 1700 for its various purposes.

Various other tools are contemplated for use with the cannula 100. Syringe-type tools are common used with microendoscopic procedures. For example, lavage system comprising a syringe insert would aid a physician deliver necessary fluids to the distal end of the system for purposes to delivering fluids to targeted cells, collecting targeted loose cells, or washing targeted cells. Syringe-type tools can easily be made to fit into the lumen tube 101.

In accordance with the present invention, a physician may use the current three part endoscopic system under various surgical exploratory procedures and other surgical procedures that require minimal invasiveness to body tissue. For example, the current invention can be used for breast tissue exploration that is normally conducted with an endoscope that passes through the breast nipple sphincter.

In the past, physicians use a fair amount of educated guessing when inserting an endoscope into breast tissue for breast exploration. Physicians have little guidance as to the location of the desired natural pathways that exist in breast tissue. Often, physicians are relegated to deducing locations of desired infected cells by manipulating the endoscope through various pathways in the breast tissue in a trial and error type method.

The current three part system utilizes its interchangeable components to guide a physician to targeted areas of the breast. Because breast infections (and cancer) often result in dilated, lactating nipples, physicians can easily locate on the surface of the nipple which sphincter to penetrate with the cannula and dilator insert. There are typically about 6-8 ducts in the human nipple that are suitable for the microendoscopic system. There is a need for a microendoscope that dilates and facilitates entry into the patient body that also allows the physician to capture images of the tissue when the microendoscope passes through the sphincter so that the physician can accurately guide the microendoscope to the targeted cells.

According to the current novel procedure, a physician will massage the chest, which typically results in lactating liquid to reach the surface of the nipple through the nipple sphincter. Then, the physician will anesthetize the nipple, normally using a local anaesthetic. At that point, the physician may enter the lactating breast sphincter using the dilator cannula 100 and the inserted dilator 200. The dilator 200 protrudes from the distal end 111 of cannula 100 exposing the tapered end, which aids the physician in passing the endoscopic system through the nipple sphincter. Once the dilator 200 and cannula 100 passes through the sphincter about 1 cm, the physician can easily remove the dilator 200 and slide in the endoscope insert 300 without removing the cannula tube 101 from the patient. The endoscope insert 300 allows for imaging at the distal end of the lumen tube so that the physician may guide the endoscope to the desired location manipulating the movement of the endoscope by viewing the images and massaging the breast.

Because the cannula 100 substantially remains in its place while the physician removes the dilator 200 and inserts the endoscope insert 300, there is minimal invasiveness to surrounding tissues. Further, because the lumen tube 101 does not have to be removed while other interchangeable components such as the endoscope insert 300 and the dilator 200 can be inserted at various times, the physician may remove the microendoscope after viewing images of certain areas in the tissue and reinsert the dilator 200 or another tool for improved access to a target area.

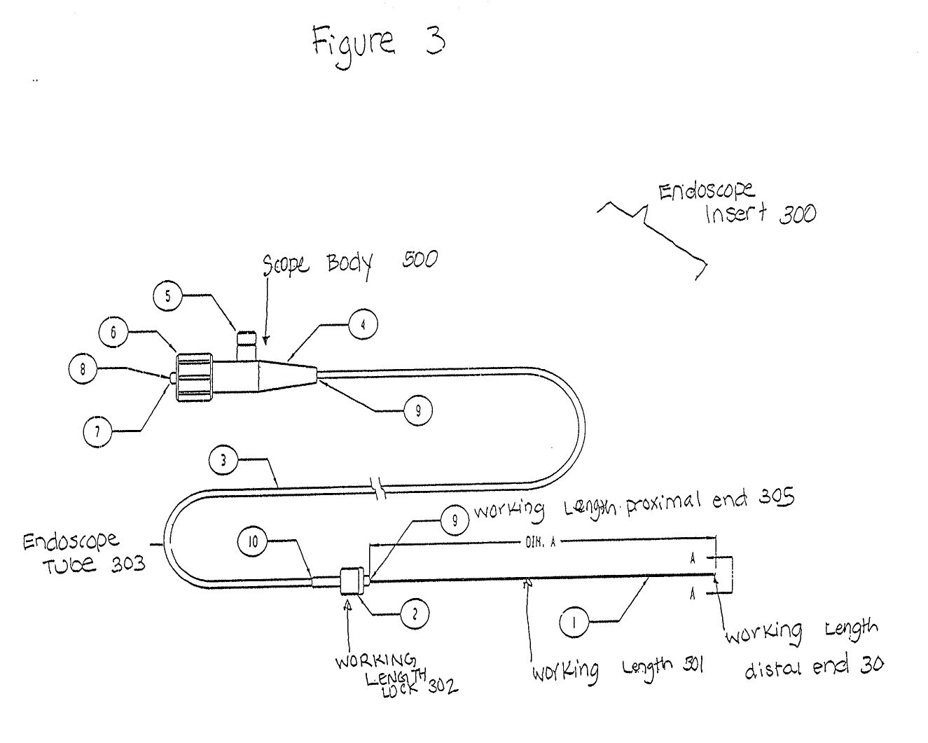

Referring now to FIG. 3, there is illustrated a side view of the endoscope insert 300. The endoscope insert 300 contains a working length 301. The endoscope insert 300 is inserted into lumen tube 101 and locked when in use. The working length 301 may have various lengths depending on the use of the device, but for purposes of breast exploration, the working length 301 may be about 10 cm long such that when the physician slides the working length 301 into the lumen tube 101, the working length distal end 304 is flush with the distal end of lumen tube 101. The working length 301 may be made from various materials including rigid plastic materials. For purposes of breast exploration, there are certain advantages of manufacturing the working length 301 from stainless steel because of its ease of manufacture and rigidity that adds support to the lumen tube 101. The working length 301 is connected to endoscope tube 303. The endoscope tube 303 may be made from various tubing materials, such as plastic tubing. The tubing is in communication with the scope body 500, which transfers the images from the illumination fibers to a video screen, such as a video monitor.

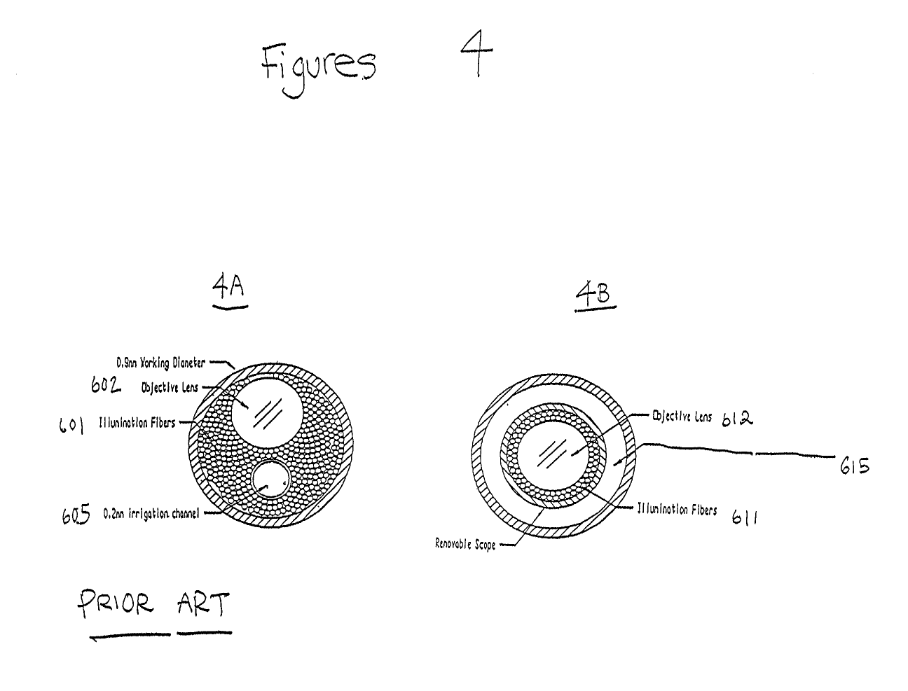

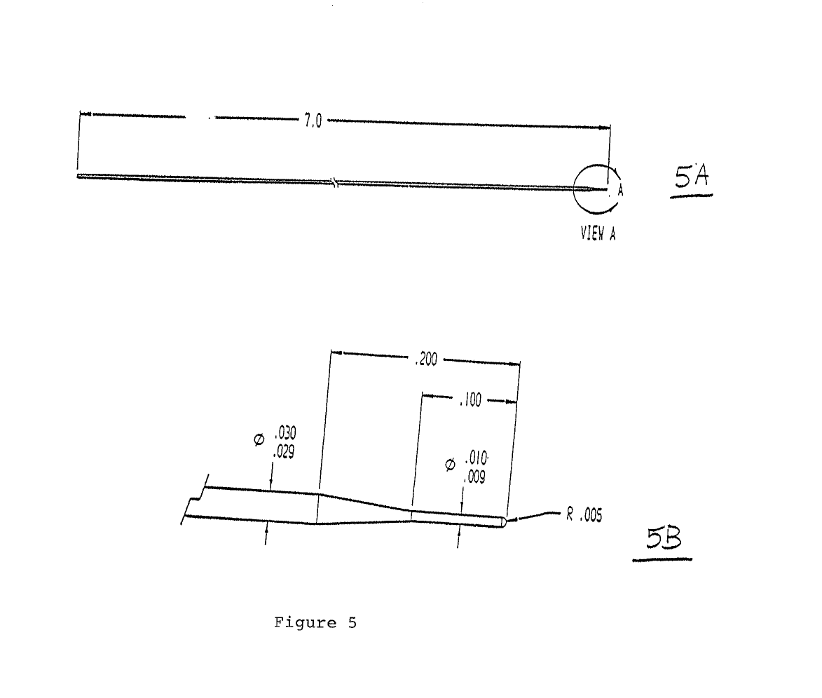

Referring to FIG. 4A, there is illustrated a cross-sectional view of the working channel of prior art endoscopes. As shown, the illumination fibers 601 surround the objective lens 602 and the irrigation channel 605. The optical bundle fibers that make up the illumination fibers 601 provide light to the distal end 111 of the endoscope. A large number of illumination fibers 601 are needed to properly light the desired area. An irrigation channel 605 is also needed to provide saline and other medication or fluids to the distal end 111. Because of the need for a large number of illumination fibers 601, the irrigation channel 605 is limited to about 0.2 mm in diameter in prior art models. Many different types of illumination fibers exist, but for purposes of the current invention, the fibers used in the current endoscope are manufactured by Fujikura or Sumitomo.

Referring to FIG. 4B, there is illustrated a cross-sectional view of the working length 301 of a current invention endoscope system. In accordance with the current invention, the number of illumination fibers is significantly reduced from prior art endoscopes. The need for the number of illumination fibers is reduced because of the less light needed with the current endoscope, which is much more narrow in a cross-sectional area than most conventional scopes. In prior art scopes, the illumination fibers simply terminate at the side arm of the endoscope inefficiently transferring light from the cable to the scope. As shown in FIG. 4B, there are a smaller number of illumination fibers 611 in the working length 301. When the removable working length 301 of the endoscope insert 300 is locked into the lumen tube 101, there exists an irrigation/working channel 615 around the outside perimeter of the endoscope insert 300.

In comparison, if the diameter of the current invention scope is the same as the prior art scope as described in FIG. 4A, the current invention would have a irrigation/working diameter of about 0.82 mm primarily because of the reduced number of illumination fibers 611. This is about 12 times larger in area than the prior art scopes if the area occupied by the objective lens 612 is held to be constant. With the greater irrigation/working area 615, the scope provides for much easier liquid delivery. In addition, lower pressure will be required to push liquid through the larger irrigation/working channel.

The objective lens 612 captures the reflected image from the light source from the illumination fibers 611. The captured image guide is transmitted from the objective lens, which is typically located at the distal end 111 to the scope body 500 through specially designed fiber optic bundles made specifically to transfer the image guide. According to the present invention, when the scope insert 300 is removed, there is a working diameter of about 0.81 mm (0.032 inches) in a 0.9 mm outside diameter lumen tube 101. This would appropriately fit the various insertable tools as discussed above.

Referring to FIGS. 5A and 5B, there is illustrated in detail the tapered distal end 204 of dilator 200. According to the current invention, the tapered end 204 can be made from various metals, alloys, or plastic materials. For purposes of a nipple dilator, the dilator can be made from stainless steel, particularly effective for its rigidity and ease of manufacturing. The platinum-tungsten alloy also provides suitable rigidity for the tip. As shown in FIG. 5A, the tip of the distal end 204, has a radius of about 0.005 mm at its end. The length of the tapered section is about 5 mm. These dimensions provide for easy entry through natural or manmade orifices, such as the nipple sphincter causing minimal damage to surrounding tissues.

In accordance with the current novel invention, the scope body 500 incorporates a fixed focus optical system. In other words, the user does not have to turn one part of the coupler to focus the image created at the distal end of the microendoscope. Instead, the scope body 500 has a fixed focus design having a predetermined focal point such that no focusing is necessary. In prior art scopes, the viewer is capable of rotating one section of the coupler to vary the distance between the lenses of the video coupler. This focusing ring allows the user to focus the coupler to produce a clear image, but because one piece of the coupler is rotatable on another, there is never a fixed orientation of one coupler section with respect to the other.

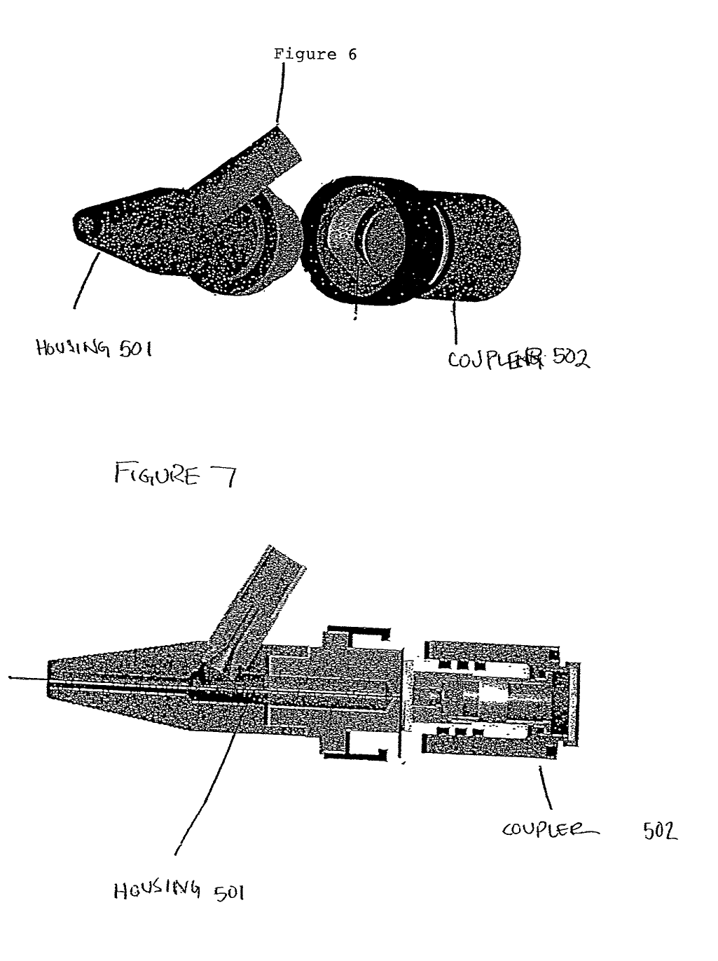

Referring now to FIG. 6, there is illustrated an angled view of the scope body 500 that connects the endoscope insert 300 to a viewing device, such as a video monitor. The endoscope tube 303 is connected to a scope body 500. The scope body 500 connects the endoscope to a camera head. The scope body 500 includes a housing 501 and coupler 502. Once the image is captured on the objective lens 612, the image is carried on optical fibers to the video coupler. The image is transferred to a CCD (Charged Coupled Device) chip 520, which is located at the end of coupler 502.

As shown in FIG. 6, the housing 501 has a light source 503, which is attached to the housing 501 by a y-connector. The housing 501 and coupler 502 is designed to connect in a very unique manner to produce fixed focus capabilities. The unique connection can take many different forms as long as the alignment of the coupler 502 and the housing 501 are established in a predetermined fashion so that there is no rotation along its common axis. In the current invention, this is accomplished by creating uniquely designed male and female portions of the coupler in the housing 501 and coupler 502.

As shown in FIG. 6, in one embodiment, the housing 501 contains a male connector having an insertable D shaped protrusion. The coupler 502 contains the respective female counterpart having a D shaped insertable section. The purpose of the D shaped parts is to provide a unique alignment so that the two opposing components, the housing 501 and coupler 502, can only fit in a predetermined orientation. Designers test the alignment of the different components, including the housing 501 and coupler 502 during manufacture of the three part microendoscopic system. By checking the image focus constantly, housing 501 and coupler 502 are aligned and marked to show what orientation would produce the best image. Once the alignment of different components are matched, the unique fitting of the coupler 502 and housing 501 are designed so that when they do fit perfectly, the alignment will be recreated.

The unique alignment of the housing 501 and coupler 502 serves many purposes. Primarily, the purpose of such alignment is used to create a stable clockwise arrangement of the housing 501 and the coupler 502. Second, because the clockwise orientation of the coupler will always be the same, there is consistency in the orientation of the distal end of the lumen tube 101 and the orientation of the scope body 500. In other words, the current invention having a fixed coupler orientation allows physicians to coordinate the lumen tube distal end 101 in a clockwise orientation by aligning the orientation with the scope body 500. For example, when a physician inserts an endoscope into a patient, the physician does not know whether the image shown from the endoscope is oriented properly or whether it is rotated clockwise or upside down, because the variable rotating mechanism of the focus does not allow for such orientation. On the other hand, when a physician uses the current system with a predetermined orientation as described in this current invention, the physician by manipulating the scope left or right can easily determine the correct orientation of the distal end 111 of the endoscope. Further, because the orientation of the scope body 500 and the orientation of the distal end are consistent, the physician can maintain its orientation alignment of the distal end throughout the surgical procedure.

To manufacture a fixed focus lens, the image guide must be transferred to the CCD chip 520 in an efficient and easily reproducible manner. This is accomplished by creating a coplanar alignment between the housing 501 and the coupler 502. As shown in FIG. 7, when the housing 501 is flush with coupler 502, the image guide of the male connector section of the housing 501 must be coplanar with the female focal plane of coupler 502. The faces of the coplanar elements must be properly aligned so that the planar faces can interact in as much of a coplanar manner as possible. In the past, there has not been a need to create such near perfect coplanar surfaces for couplers because a physician user was able to vary the focal points of the image. To make a smooth surface, the manufacturer must polish the front of the coupler 502 using a novel method in the endoscopic field.

As shown in FIG. 13, the image guide is transferred from the distal end of the cannula 101 to the housing 501. Before the coupler 502 is polished, the coupler 502 is manufactured to have extra polishing material on its polishing surface. In other words, the coupler 502 contains extra backing on its male connector component so that when the backing is polished, the polishing does not penetrate into the inner parts of the coupler 502.

Polishing is performed using a lapping film, which acts much like sandpaper. Conventional lapping films made from diamond and aluminum oxide may be used. Lapping films come in various polishing grades of fineness. The surface of the housing is first polished using a low grade lapping paper, such as 25 micron lapping paper. Gradually, finer and finer lapping paper is used until the surface is adequately polished. In one embodiment, the surface is polished ultimately using 0.3 micron lapping paper. The same is done to the female connector component of the coupler 502. These smooth polishing surfaces provide the transfer of the image guide.

In one manufacturing method, the polishing surface of coupler 502 is designed with triangular shaped grooves or other shaped grooves that are designed to efficiently polish non-flat surfaces. These differently shaped grooves aid in the polishing of uniquely shaped components, such as the D-shaped male connectors on the coupler 502. The irregular shaped grooves aid in the consistent polishing of the surface by preventing any type of hydroplaning the lapping paper may experience during the polishing phase of the unique components. The grooves provide certain water channels to prevent hydroplaning, similar to the tread of a tire.

Once the two sections are polished, the connection means are connected in its uniquely designed orientation. There are various methods by which to connect the housing 501 and the coupler 502. One method is to connect the two components using conventional screw type locking mechanisms as shown in FIG. 6. There are other luer lock and luer slip configurations for the proper alignment. There are other snap-on type connection methods to maintain the orientation of the components. Other unique shapes can properly align housing 501 and coupler 502, without the use of a D-shape configuration. It may have a trapezoidal configuration or any other type of non-circular configuration, shape, or design such that the connection orientation may only produce one proper clockwise orientation.

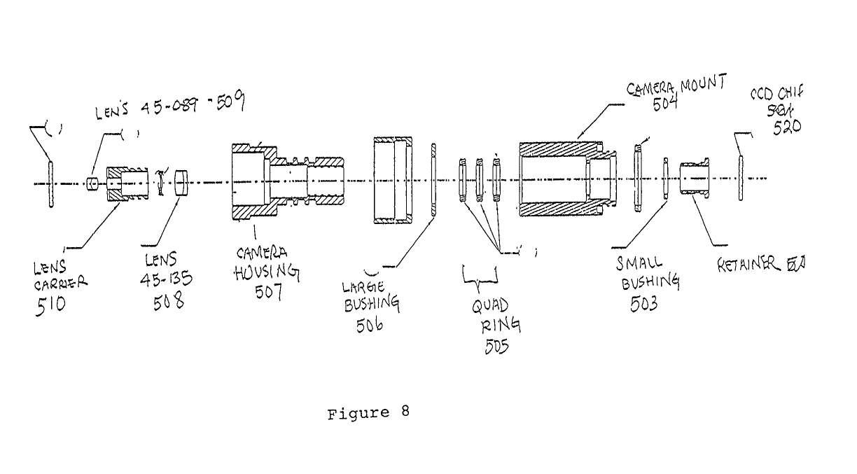

In reference to FIG. 8, there is illustrated a layout of the other conventional components of the scope body 500. Lens carrier 510 holds two lenses 509 and 510 in a predetermined distance from each other to create a certain magnification. The image guide is passed through these lenses that is located in the camera housing 507. The large bushing 506 and the quad rings 505 are connected to the camera mount 504 and the small bushing 503. The large bushing 506 and small bushing 503 are aluminum anodes used in transferring the image guide to the CCD chip 520. The camera mount 504 is attached to the CCD chip 520 and a camera (not shown). The components in the scope body 500 passes the image guide to the CCD chip in a fixed focus manner without the need to manually focus the image.

I claim:

1. An endoscope system comprising:
a) a cannula having a uniform lumen comprising a tubular member with a predetermined length, outside diameter and inside diameter, said cannula having a proximal end, a distal end, and a lumen extending therebetween, said cannula having a cross section area,
b) a dilator having a wire frame with a predetermined length greater than said length of said cannula, an outer diameter that has a cross-sectional area that is smaller than said cross section area of said lumen such that said dilator can slide longitudinally and coaxially through said cannula,
c) a scope assembly comprising a tubular member with a certain length, outside diameter and inside diameter, said tubular member having a proximal end, a distal end, and a lumen therebetween, said scope assembly having a scope operatively connected to said proximal end of said tubular member, and optical fibers extending from said scope to said distal end of said tubular member,
d) said lumen containing optical fibers for transmitting light from said proximal end of said cannula to said distal end of said cannula,
e) and a hub attached to said cannula having a bore that is operatively connected to said lumen of the said cannula.
f) an apparatus operatively connected to said scope assembly for viewing said image
g) a twist window system comprising a first twist window tubular member with a certain length, outside diameter and inside diameter, said first twist window tubular member having a proximal end, a distal end, and a uniform lumen therebetween, said twist window tubular member being sized to fit within said cannula coaxially,
a second twist window tubular member having a certain length, outside diameter and inside diameter, said second twist window tubular member being sized to fit within and insertable within said first twist window tubular member, said first twist window tubular member having a first cutting surface created by an aperture or window, said second twist window tubular member having a second cutting surface created by an aperture or window,
h) a scissors-like insert having a first scissors tubular member with a certain length, outside diameter and inside diameter, said first scissors tubular member having a proximal end, a distal end, and a uniform lumen therebetween, said scissors tubular member being sized to fit within said cannula coaxially, a second scissors tubular member having a certain length, outside diameter and inside diameter, said second scissors tubular member being sized to fit within and insertable within said first scissors tubular member, said first scissors tubular member having a cutting surface created by an edge at said distal end of said first tubular member, said second tubular member having a second cutting surface created by an aperture or window, said first and second cutting surfaces having a predetermined locations such that when second scissors tubular member is inserted within said first tubular member, said first and second cutting surfaces are aligned,
i) a tissue abrader insert comprising a tissue abrader first tubular member with a certain length, outside surface, outside diameter and inside diameter, said tissue abrader first tubular member having a proximal end, a distal end, and a uniform lumen therebetween, said tissue abrader tubular member being sized to fit within said cannula coaxially, said tissue abrader tubular member having burrs created by small protrusions on said surface of said tissue abrader first tubular member, said protrusions having small openings and cutting edges sized to cut and fit small tissue cells,
j) a spring grasp insert comprising a spring grasp first tubular member with a certain length, outside surface, outside diameter and inside diameter, said spring grasp first tubular member having a proximal end, a distal end, and a uniform lumen therebetween, said spring grasp tubular member being sized to fit within said cannula coaxially, a grasp tool comprising an elongated member with a proximal end and a distal end, said grasp tool having at least two gripping jaws capable of closing and opening and collecting targeted tissue cells, said gripping jaws being located at the distal end of said grasp tool, said gripping jaws having a predetermined shape in an open position.
k) a laser insert comprising a laser tubular member with a certain length, outside surface, outside diameter and inside diameter, said laser tubular member having a proximal end, a distal end, and a uniform lumen therebetween, said tubular member being sized to fit within said cannula coaxially, a laser transmitting fiber extending from said proximal end to said distal end capable of transmitting various types of laser light to the distal end of said laser insert system, and a video coupler comprising a housing having a light source and a light path that is transferable to the distal end of the endoscope, said housing having a connection means, a housing with connection means that fit said connection means of said housing so that there is only one optical orientation by which they can connect, lens means mounted within the housing for magnifying an image from the endoscope in order to provide a magnified image, means for aligning the housing and the hub in a predetermined orientation so that it produces an fixed focus image.

2. An endoscope system comprising:
a) a cannula having a uniform lumen comprising a tubular member with a predetermined length, outside diameter and inside diameter, said cannula having a proximal end, a distal end, and a lumen extending therebetween, said cannula having a cross section area,
b) a dilator having a wire frame with a predetermined length greater than said length of said cannula, an outer diameter that has a cross-sectional area that is smaller than said cross section area of said lumen such that said dilator can slide longitudinally and coaxially through said cannula,
c) a scope assembly comprising a tubular member with a certain length, outside diameter and inside diameter, said tubular member having a proximal end, a distal end, and a lumen therebetween, said scope assembly having a scope operatively connected to said proximal end of said tubular member, and optical fibers extending from said scope to said distal end of said tubular member,
d) said lumen containing optical fibers for transmitting light from said proximal end of said cannula to said distal end of said cannula,
e) and a hub attached to said cannula having a bore that is operatively connected to said lumen of the said cannula.

3. The endoscope system of claim 2 further comprising a dilator with a proximal end and a distal end, said distal end of dilator being tapered to a point for entry through small body orifices, such as the nipple sphincter.

4. The endoscope system of claim 2 further comprising a dilator with a proximal end and a distal end, said dilator having a dilator cap attached to said proximal end of said dilator, said dilator cap having a locking mechanism so that when the cap is locked with said cannula, said end of dilator protrudes from the distal end of said cannula.

5. An endoscope system comprising:
a) a cannula comprising a tubular member with a certain length, outside diameter and inside diameter, said cannula having a proximal end, a distal end, and a lumen extending therebetween, said cannula having a cross section area,
b) a dilator having a wire frame with a predetermined length greater than said length of said cannula, an outer diameter that has a cross-sectional area that is smaller than said cross section area of the lumen such that said dilator can slide longitudinally and coaxially through said cannula,
c) a scope assembly comprising a tubular member with a certain length, outside diameter and inside diameter, said tubular member having a proximal end, a distal end, and a lumen therebetween, said scope assembly having a scope operatively connected to said proximal end of said tubular member, and optical fibers extending from said scope to said distal end of said tubular member, said scope assembly capable of producing an image at the distal end of said tubular member that is capable of being transmitted through said optical fibers of said tubular member,
d) said lumen containing optical fibers for transmitting light from a light source near said proximal end of said cannula to said distal end of said cannula,
e) and a hub attached to said cannula having a bore that is in operatively connected to said lumen of the said cannula.
f) an apparatus operatively connected to said scope assembly for viewing said image.

6. The endoscope system of claim 5 further comprising a viewing image means comprising a monitor for depth of field viewing having a transparent screen and said optical element positioned between said transparent screen and the viewer.

7. A twist window insert system comprising
a cannula having a uniform lumen,
a first tubular member with a certain length, outside diameter and inside diameter, said first tubular member having a proximal end, a distal end, and a uniform lumen therebetween, said tubular member being sized to fit within said cannula coaxially,
a second tubular member having a certain length, outside diameter and inside diameter, said second tubular member being sized to fit within and insertable within said first tubular member,
said first tubular member having a first cutting surface created by an aperture or window,
said second tubular member having a second cutting surface created by an aperture or window,
said first and second cutting surfaces having a predetermined location such that when second tubular member is inserted within said first tubular member, said first and second cutting surfaces are aligned.

8. The twist window system of claim 7 further comprising the capability of twisting said second tubular member within said first tubular member when the second tubular member is inserted into the first tubular member such that the aligned windows are capable of pinching off the targeted tissue cells.

9. The twist window system of claim 8 further comprising an endoscope component extending longitudinally within said second tubular member and capable of producing an image at the distal end of said twist window and capable of being transmitted to the proximal end of said twist window.

10. A scissors-like insert system comprising
a cannula having a uniform lumen,
a first tubular member with a certain length, outside diameter and inside diameter, said first tubular member having a proximal end, a distal end, and a uniform lumen therebetween, said tubular member being sized to fit within said cannula coaxially,
a second tubular member having a certain length, outside diameter and inside diameter, said second tubular member being sized to fit within and insertable within said first tubular member,
said first tubular member having a first cutting surface created by an edge at said distal end of said first tubular member,
said second tubular member having a second cutting surface created by an aperture or window,
said first and second cutting surfaces having a predetermined locations such that when second tubular member is inserted within said first tubular member, said first and second cutting surfaces are aligned.

11. The scissors-like system of claim 10 further comprising the capability of sliding said second tubular member within said first tubular member when the second tubular member is inserted into the first tubular member such that the cutting surfaces are capable of shredding targeted tissue cells.

12. The scissors-like system of claim 10 further comprising an endoscope component extending longitudinally within said second tubular member and capable of producing an image at the distal end of said scissors-like system and capable of being transmitted to the proximal end of said scissors-like system.

13. A tissue abrader insert system comprising
a cannula having a uniform lumen,
a first tubular member with a certain length, outside surface, outside diameter and inside diameter, said first tubular member having a proximal end, a distal end, and a uniform lumen therebetween, said tubular member being sized to fit within said cannula coaxially,
said tubular member having burrs created by small protrusions on said surface of said first tubular member,
said protrusions having small openings and cutting edges sized to cut and fit small tissue cells.

14. The tissue abrader system of claim 13 further comprising an endoscope component extending longitudinally within said tubular member and capable of producing an image at the distal end of said tissue abrader system and capable of being transmitted to the proximal end of said system.

15. A sprig grasp insert system comprising
a cannula having a uniform lumen,
a first tubular member with a certain length, outside surface, outside diameter and inside diameter, said first tubular member having a proximal end, a distal end, a uniform lumen therebetween, said tubular member being sized to fit within said cannula coaxially,
a grasp tool comprising an elongated member with a proximal end and a distal end,
said grasp tool having at least two gripping jaws capable of closing and opening and collecting targeted tissue cells,
said gripping jaws being located at the distal end of said grasp tool.
said gripping jaws having a predetermined shape in an open position.

16. The spring grasp system of claim 15 further comprising the capability of sliding said first tubular member with said grasp tool such that when said grasp tool is pulled within said first tubular member, said grasp tool closes said gripping jaws capable of collecting targeted cells.

17. The spring grasp system of claim 16 further comprising an endoscope component extending longitudinally within said tubular member and capable of producing an image at the distal end of said spring grasp system and capable of being transmitted to the proximal end of said system.

18. The spring grasp system of claim 15 further comprising grasp tools that are made from memory metal.

19. The spring grasp system of claim 15 further comprising an elbow located substantially in the middle of each said gripping jaws, said elbow capable of bending such that when said grasping tool is pulled within said outer tube, said elbows bend capable of collecting targeted cells.

20. An laser insert system comprising
a cannula having a uniform lumen,
a tubular member with a certain length, outside surface, outside diameter and inside diameter, said first tubular member having a proximal end, a distal end, and a uniform lumen therebetween, said tubular member being sized to fit within said cannula coaxially,
a laser transmitting fiber extending from said proximal end to said distal end capable of transmitting various types of laser light to the distal end of said laser insert system.

21. The laser system of claim 20 further comprising an endoscope component extending longitudinally within said tubular member and capable of producing an image at the distal end of said laser system and capable of being transmitted to the proximal end of said system.

22. An endoscope system comprising:
a) a cannula comprising a tubular lumen with a predetermined length, outside diameter and inside diameter, said cannula having a proximal end, a distal end, and a lumen extending therebetween, said cannula having a cross section area of the lumen,
c) a scope assembly comprising a tubular member with a certain length, outside diameter and inside diameter, said tubular member having a proximal end, a distal end, and a lumen therebetween, said scope having illumination fibers extending therethrough,
d) said objective lens extending centrally throughout the scope assembly, said illumination fibers surround said objective lens such that the objective lens is substantially located in the center of the scope assembly,
e) said cannula being sized such that when the scope assembly is inserted into said cannula, there is created room between said scope assembly and inside surface of said cannula so that there is an irrigation area capable of delivering various liquids to targeted areas.

23. An endoscope system comprising:
(a) a cannula means for insertion into the human body having a predetermined length comprising a open proximal end, a open distal end and a lumen extending therebetween, said lumen having an inside diameter and an outside diameter, said outside diameter capable of passing through the sphincter of a nipple,
(b) a dilator means for insertion into cannula means and for assisting physicians guide the cannula means to desired locations in the body tissue,
(c) an endoscope means for imaging desired tissue cells in the body and producing an image for physicians.

24. A coupler device for coupling an endoscope to a camera or display means comprising:
a housing having a light source and a light path that is transferable to the distal end of the endoscope, said housing having a connection means,
a housing with connection means that fit said connection means of said housing so that there is only one optical orientation by which they can connect,
lens means mounted within the housing for magnifying an image from the endoscope in order to provide a magnified image,
means for aligning the housing and the hub in a predetermined orientation so that it produces an fixed focus image.

25. The device of claim 24 wherein:
said connection means of said housing is a male connector in the shape of the letter “D”,
said connection means of said housing is a female connector in the shape of the letter “D”.

26. The device of claim 24 wherein said hub has a flat back surface and where the housing has a flat front surface so that when the housing and hub are connected, they share a flush, coplanar surface between the image guide and the focal plane.

27. A method of manufacturing an endoscopic system comprising the steps of:
a) making a cannula comprising a tubular member with a certain length,
b) making dilator having a wire frame such that said dilator can slide longitudinally and coaxially through said cannula,
c) making a coupler to connect the endoscope to the video viewing device,
d) polishing parts of the coupler to produce flat surfaces such that the image guide of the housing is flush and flat against the front planar surface of the housing so that focal point is fixed between the transfer of image from the hub to the housing.

28. A method of performing an endoscopic procedure on a patient's breast comprising the steps in order of:
a) using a substantially semi-rigid dilator and cannula to pass through a sphincter in the breast nipple,
b) using the dilator to guide the cannula to a desired area of breast tissue,
c) removing the dilator,
d) inserting an endoscopic assembly into said cannula,
e) taking images of cells at the distal end of the endoscope,
f) further guiding the endoscope insert to desired target cells using the images created by the endoscope.

29. The method of performing an endoscopic procedure on a patient's breast comprising the step of using a fixed focus video coupler incapable of varying the focal point so that the when the endoscope is inserted into a patient, a physician is capable of determining the clockwise orientation of the endoscope.
